(19) United States
(12) Patent Application Publication
(10) Pub. No.: US 2017/0015811 A1
(43) Pub. Date: Jan. 19, 2017

(54) HYDROGEN SULFIDE SCAVENGER

(71) Applicants: JEAN VALERY MARTIN, PRINCETON, NJ (US); GIOVANNI ONNEMBO, WAYNE, NJ (US)

(72) Inventors: JEAN VALERY MARTIN, PRINCETON, NJ (US); GIOVANNI ONNEMBO, WAYNE, NJ (US)

(73) Assignee: Innophos Inc., Cranbury, NJ (US)

(21) Appl. No.: 15/262,438

(22) Filed: Sep. 12, 2016

Related U.S. Application Data
(63) Continuation-in-part of application No. 14/170,224, filed on Jan. 31, 2014, now Pat. No. 9,441,092.

(51) Int. Cl. C08K 5/17; C07C 227/16

(52) U.S. Cl. CPC C08K 5/175; C07C 227/16

(57) ABSTRACT

The present disclosure is directed to a composition comprising asphalt and an amino acid metal chelate.

### CROSS REFERENCE TO RELATED APPLICATIONS

The present application is a continuation-in-part of U.S. Ser. No. 14/170,224, filed Jan. 31, 2014, the entire contents of which are hereby incorporated by reference.

### FIELD OF THE INVENTION

The present disclosure relates to a hydrogen sulfide scavenger for use as an additive in asphalt.

### BACKGROUND OF THE INVENTION

Asphalt is commonly used in the construction and paving of roads. Asphalt is a mixture of aggregate material, such as sand, gravel, and crushed stone, with hot bitumen. The bitumen coats the aggregate material to give the asphalt, which may be spread as a uniform layer upon a road bed, and compacted and smoothed with heavy rolling equipment.

Asphalt invariably contains sulfur. The amount of sulfur will depend on the origin of the crude oil, as well as the processes used to refine the crude oil, into asphalt. The sulfur may exist in different forms. For example, sulfur may be in the form of hydrogen sulfide. Hydrogen sulfide, or dihydrogen sulfide, is a chemical compound with the formula H₂S. It is ,a colorless, poisonous, flammable gas with the characteristic foul odor.

Hydrogen sulfide may be released form asphalt, in particular when the asphalt is heated to a certain temperature. For example, hydrogen sulfide results from the dehydrogenation reactions that occur between bitumen and sulfur at the hot mixing temperatures, e.g. temperatures greater than 140° C. Hydrogen sulfide emissions are regulated. Therefore, there exists a need to reduce the amount of hydrogen sulfide in asphalt. Accordingly, the present disclosure provides for a reduced or low release of hydrogen sulfide during the preparation of asphalt, as well as in the final asphalt material.

### SUMMARY OF THE INVENTION

The present disclosure is related to a family of metals chelates for use as a hydrogen sulfide scavenger in asphalt, and the preparation thereof. The metal chelates, in particular amino acid metal chelates, are particularly efficient at reducing the hydrogen sulfide emissions of asphalt.

The present disclosure is directed to a composition comprising asphalt and an amino acid metal chelate.

The present disclosure is directed to a method to produce the composition is by the reaction of copper carbonate and glycine water, wherein an effective amount of copper carbonate exists after the production of the amino acid chelate sufficient to reduce hydrogen sulfide emission.

The present disclosure is also directed to a method of reducing hydrogen sulfide emission from a substance that emits hydrogen sulfide by combining an amino acid metal chelate tri asphalt, or an asphalt mix.

### DETAILED DESCRIPTION OF THE INVENTION

The present disclosure is directed to a composition comprising asphalt and an amino acid metal chelate. The composition is produced by the reaction of copper carbonate and glycine in waterₛ wherein an effective amount of copper carbonate exists after the production of the amino acid chelate sufficient to reduce hydrogen sulfide emission. Obtaining the composition by this method provides a commercially viable composition which has unique characteristics, and in addition, residual copper carbonate that provides a “separate” hydrogen sulfide reducing agent.

The amino acid metal chelate may also be selected form the following: Boron Amino Acid Chelate; Boron Aspartate; Boron Citrate; Boron Glycinate; Calcium Alphaketoglutarate; Calcium Amino Acid Chelate; Calcium Arginate; Calcium Ascorbate; Calcium Aspartate; Calcium Caprylate; Calcium Carbonate; Calcium Citrate Malate; Calcium Glycinate; Calcium D-Glucarate; Calcium Krebs Cycle; Calcium Lactate; Calcium Malate; Calcium Orotate; Calcium Succinate; Chromium Amino Acid Chelate; Chromium Arginate; Chromium Chloride; Chromium Dinicotinate/Glycinate; Chromium Picolinate; Chromium Nicotinate; Chromium Trit; Chromium Yeast; Chromium Nicotinate/Glycinate; Copper Amino Acid Chelate; Copper Aspartate; Copper Carbonate; Copper Citrate; Copper Gluconate; Copper Glycinate; Copper Sulfate; Copper Yeast; Iron Amino Acid Chelate; iron Aspartate; iron Bis-Glycinate HCl Soluble; Iron Citrate; Iron Fumarate; Iron Gluconate; Iron Glycinate; Iron Sulfate; Iron Yeast; Lithium Aspartate; Lithium Orotate; Magnesium Alphaketoglutarate; Magnesium Amino Acid Chelate; Magnesium Ascorbate; Magnesium Aspartate; Magnesium Citrate; Magnesium Gluconate; Magnesium Glycinate; Magnesium Malate; Magnesium Orotate; Magnesium Oxide; Magnesium Succinate; Magnesium Taurinate; Magnesium Yeast; Manganese Amino Acid Chelate; Manganese Aspartate; Manganese Carbonate; Manganese Citrate; Manganese Gluconate; Mananese Glycinate; Manganese Sulfate; Manganese Yeast; Molybdenum Amino Acid Chelate; Molybdenum Trit; Molybdenum Yeast; Sodium Molybdate; Phosphorus Amino Acid Chelate; Dicalcium Phosphate; Potassium Amino Acid Chelate; Potassium Ascorbate; Potassium Aspartate; Potassium Citrate; Potassium Chloride; Potassium D-Glucarate; Potassium Gluconate; Potassium Glycerophosphate; Potassium Iodide Trit; Potassium Succinate; Selenium Amino Acid Chelate; Selenium Aspartate; L-Selenomethionine; Selenium Yeast; Sodium Selenate; Sodium Selenite; Strontium Aspartate; Strontium Citrate; Strontium Glycinate; Vanadium Amino Acid Chelate; Vanadium Citrate; Bis-Maltolato Oxo Vanadium; Vanadyl Sulfate; Sodium Metavanadate; Zinc Acetate; Zinc Arginate; Zinc Amino Acid Chelate; Zinc Ascorbate; Zinc Aspartate, Zinc Gluconate; Zinc Glycinate; Zinc Methionate; Zinc Oxide; Zinc Picolinate; Zinc Sulfate; and Zinc Yeast

The present disclosure is directed to a method to produce the composition is by the reaction of copper carbonate and glycine in water, wherein an effective amount of copper carbonate exists after the production of the amino acid chelate sufficient to reduce hydrogen sulfide emission.

The composition is produced by the reaction of copper carbonate and glycine in water wherein an effective amount of copper carbonate exists after the production of the amino acid chelate sufficient to reduce hydrogen sulfide emission. Obtaining the composition by this method provides a commercially viable composition which has unique characteristics (as provided herein), and in addition, residual copper carbonate that provides a “separate” hydrogen sulfide reducing agent.

Applicant's expertise and investigation in the area of the present invention recognizes that decreased (low) particle size, resulting in lower bulk density Cu Carb provides beneficial characteristics, which are important to the commercial issues for the “scavenger” concept. Most particularly, low particle size and therefore, there is provides more surface area available for a reaction to occur. Below is the particle size data for the present invention; the values are in microns.

| Sample Name | d (0.1) | d (0.5) | d (0.9) |
|---|---|---|---|
| High Density Copper Carbonate Average | 11.1 | 19.513 | 33.88 |
| Low Density Copper Carbonate Average | 0.997 | 2.498 | 11.937 |
| Copper Bisglycinate | 1.291 | 3.445 | 15.136 |

Referring to pore size, increased pore size provides additional surface area resulting in improved reactivity (it is not only particle size that contributes to surface area). The decreased particle size copper carbonate results in higher specific surface area; thus defining a inversely proportional relationship As an advantage to commercialization, Applicants have discovered by dry milling to smaller particle size the bulk density would decrease. One skilled in the art would appreciate pore void volume fraction can be calculated by means of commonly used analytical equipment with specific purpose of calculating pore size and volume as well as particle size.

The disclosure is also directed to a method to reduce hydrogen sulfide emissions by adding the composition of the present invention to a substance that emits hydrogen sulfide.

### EXAMPLES

Example 1. Hydrogen sulfide emissions were measured from asphalt samples containing an amino acid metal chelate versus a control containing no amino acid metal chelate. Three asphalt samples were prepared and their hydrogen sulfide emissions measured after 1 hour in storage. To two samples, 0.5% amino acid metal chelate additive was added, CuGlyc (copper bis-glycinate) and ZnGlyc (zinc bis-glycinate) respectively. The hydrogen sulfide emissions were measured again after 5 minutes and 1 hour. Table 1 lists the results. The addition of the amino acid metal chelate showed significant reduction in hydrogen sulfide emissions.

TABLE 1
Hydrogen Sulfide Emission

| | | | | | H₂S (ppm) 1 |
|---|---|---|---|---|---|
| | | | H₂S (ppm) | H₂S (ppm) 5 | hour after |
| | | | Storage | minutes after | adding |
| % | | | at 160 | addition of | scavenger |
| Additive | | Temp | one hour | scavenger | material |
| 0 | Control | 180° C. | 16 | | 10 |
| 0.5 | CuGlyc | 180° C. | 12 | 4 | 1 |
| 0.5 | ZnGlyc | 180° C. | 10 | 5 | 1 |

Example 2. Additional amino acid metal chelates that may be used in asphalt, or the preparation thereof are listed m Table 2.

TABLE 2
Amino Acid Metal Chelates

| Metal | Chalating agent |
|---|---|
| Chromium | Amino Acid |
| | Arginate Chloride |
| | Dinicotinate/Glycinate |
| | Picolinate |
| | Nicotinate |
| | TritChromium Yeast |
| Copper | Amino Acid |
| | Aspartate |
| | Carbonate |
| | Citrate |
| | Gluconate |
| | Sulfate |
| | Yeast |
| Iron | Amino Acid |
| | Aspartate |
| | Bis-Glycinate |
| | Citrate |
| | Fumarate |
| | Gluconate |
| | Sulfate |
| | Yeast |
| Manganese | Amino Acid |
| | Aspartate |
| | Carbonate |
| | Citrate |
| | Gluconate |
| | Sulfate |
| | Yeast |
| Molybdenum | Amino Acid |
| | TritMolybdenum |
| | Yeast |
| | Sodium Molybdate |
| Selenium | Amino Acid |
| | Aspartate |
| | L-Selenomethionine |
| | Yeast |
| | Sodium Selenate |
| | Sodium Selenite |
| Strontium | Aspartate |
| | Citrate |
| | Glycinate |
| Vanadium | Amino Acid |
| | Citrate |
| | Bis-Maltolato Oxo Vanadium |
| | Vanadyl Sulfate |
| | Sodium Metavanadate |
| Zinc | Acetate |
| | Arginate |
| | Amino Acid |
| | Ascorbate |
| | Aspartate |
| | Gluconate |
| | Glycinate |
| | Methionate |
| | Picolinate |
| | Sulfate |
| | Yeast |

1. A composition comprising:
asphalt or an asphalt mix; and
an amino acid chelate produced by the reaction of copper carbonate and glycine in water, wherein, after the production of the amino acid chelate, an effective amount of copper carbonate remains able to reduce hydrogen sulfide emission.

2. The composition of claim 1, wherein the composition is devoid of water.

3. The composition of claim 1, wherein the particle size of the copper carbonate is from about 0.99 microns to about 11.0 microns.

4. The composition of claim 1, wherein the composition has an increased reactivity area inversely proportional to the particle size of the reduced density copper carbonate.

5. A method of producing a hydrogen sulfide scavenger comprising the steps of:
producing an amino acid chelate by the reaction of copper carbonate and glycine in water,
wherein an effective amount of copper carbonate exists after the production of the amino acid chelate in an amount sufficient to reduce hydrogen sulfide emissions.

6. A method of reducing hydrogen sulfide emissions comprising:
preparing the hydrogen sulfide scavenger of claim 1; and
mixing the hydrogen sulfide scavenger with a composition that emits hydrogen sulfide.
